(19) United States
(12) Patent Application Publication
(10) Pub. No.: US 2010/0016648 A1
(43) Pub. Date: Jan. 21, 2010

(54) METHOD FOR INCREASING YIELDS OF ETHYLENE AND PROPYLENE IN MTO PROCESS

(75) Inventors: Guozhen Qi, Shanghai (CN); Siqing Zhong, Shanghai (CN); Yuanfei Yang, Shanghai (CN); Huawen Wang, Shanghai (CN)

Correspondence Address: SCULLY SCOTT MURPHY & PRESSER, PC, 400 GARDEN CITY PLAZA, SUITE 300, GARDEN CITY, NY, 11530, US

(73) Assignee: CHINA PETROLEUM & CHEMICAL CORPORATION, Beijing (CN)

(21) Appl. No.: 12/526,406

(22) PCT Filed: Feb. 5, 2008
(86) PCT No.: PCT/CN2008/000328

§ 371 Date: Sep. 3, 2009

(30) Foreign Application Priority Data
Feb. 7, 2007 (CN) 200710037231.X

(51) Int. Cl. C07C 1/00

(52) U.S. Cl. 585/639

(57) ABSTRACT

The present invention discloses a method for enhancing yields of ethylene and propylene in MTO process, comprising: i) feeding a feedstock comprising C₄ hydrocarbon and at least one of methanol and dimethyl ether from a distributor at the bottom of a reactor and optionally from at least one location above the distributor into a reaction zone containing a molecular sieve catalyst; ii) allowing the feedstock to react in the presence of the molecular sieve catalyst, to form a product stream comprising ethylene, propylene and C₄ hydrocarbon; iii) withdrawing the product stream from the top of the reactor, and passing it to a separation system, to separate ethylene, propylene and C₄ hydrocarbon; and iv) circulating the C₄ hydrocarbon separated in step iii) back to step i).

### CROSS REFERENCE OF RELATED APPLICATIONS

The present application claims the benefit of the Chinese Patent Application No. 200710037231.X, filed on Feb. 7, 2007, which is incorporated herein by reference in its entirety and for all purposes.

### FIELD OF THE INVENTION

The present invention relates to a method for increasing yields of ethylene and propylene in MTO process.

### BACKGROUND OF THE INVENTION

Light olefins, defined as ethylene and propylene in the present invention, are important basic chemical feedstock, and the demand for them is increasing. At present, ethylene and propylene are mainly produced from petroleum feedstock by catalytic cracking or steam cracking. However, as petroleum resources are being exhausted and their prices are rising increasingly, other approaches for producing ethylene and propylene are paid more and more attention.

An important approach for producing light olefins from non-petroleum feedstock is the conversion of oxygenates, such as lower alcohols (methanol, ethanol), ethers (dimethyl ether, methyl ethyl ether), esters (dimethyl carbonate, methyl formate) and the like to olefins, especially the conversion of lower alcohols to light olefins. The production of light olefins from methanol or dimethyl ether is a promising process, because methanol can be produced in large scale from coal or natural gas via syngas.

Many processes for converting an oxygenate to light olefins, in particular MTO process, have been disclosed in literatures. For example, U.S. Pat. No. 6,166,282 discloses a technique for converting an oxygenate to light olefins, wherein a fast fluidized-bed reactor is employed, and gaseous feedstock is passed at a lower gas velocity through a dense phase reaction zone and then enters upwards a fast separation zone having a rapidly reduced internal diameter, where most of entrained catalyst is preliminarily separated by a specific gas-solid separation means.

CN1723262 discloses a multiple riser reaction apparatus with centralized catalyst return useful in a process for converting an oxygenate to light olefins, which apparatus comprises a plurality of riser reactors, a gas-solid separation zone, a plurality of deviating members, etc., wherein each of the riser reactors has an end into which a catalyst is fed, and the riser reactors converge at the separation zone, where the catalyst is separated from the product gas.

Although many investigations on MTO process have been accomplished, there is still a need for a method which can give further enhanced yields of ethylene and propylene at lower costs.

### SUMMARY OF THE INVENTION

The present inventors have made diligently studies and, as a result, they have found that in a conversion process using methanol and/or dimethyl ether as feedstock, C₄ hydrocarbon may also be effectively converted to light olefins under selected conditions and, at the same time, C₄ hydrocarbon further serves as a diluent, thereby enhancing the selectivity of methanol and/or dimethyl ether to olefins. Based on this find, the present invention has been made.

An object of the invention is to provide a method for enhancing yields of ethylene and propylene in MTO process, comprising: i) feeding a feedstock comprising C₄ hydrocarbon and at least one of methanol and dimethyl ether from a distributor at the bottom of a reactor and optionally from at least one location above the distributor into a reaction zone containing a molecular sieve catalyst; ii) allowing the feedstock to react in the presence of the molecular sieve catalyst, to form a product stream comprising ethylene, propylene and C₄ hydrocarbon; iii) withdrawing the product stream from the top of the reactor, and passing it to a separation system, to separate ethylene, propylene and C₄ hydrocarbon; and iv) circulating the C₄ hydrocarbon separated in step iii) back to step i).

Since there is a comparatively large amount of C₄ hydrocarbon feedstock to be further processed in worldwide range and the reaction process for converting methanol and/or dimethyl ether to light olefins can also produce a significant amount of mixed C₄ hydrocarbons (of which yield based on carbon is generally about 10 wt %, and of which more than 90 wt % is olefins, predominately 1-butene and 2-butene), the conversion of C₄ hydrocarbon to more valuable ethylene and propylene in MTO process will markedly enhance the economics of the whole process.

### BRIEF DESCRIPTION OF THE DRAWINGS

The above object as well as other objects of the present invention will be apparent from the following detailed description on the present invention with reference to the drawings, wherein

FIG. 1 is a schematic of an embodiment of the reactors useful in the method of the present invention.

### DETAILED DESCRIPTION OF PREFERRED EMBODIMENTS

The present invention provides a method for enhancing yields of ethylene and propylene in MTO process, comprising:

i) feeding a feedstock comprising C₄ hydrocarbon and at least one of methanol and dimethyl ether from a distributor at the bottom of a reactor and optionally from at least one location above the distributor into a reaction zone containing a molecular sieve catalyst;

ii) allowing the feedstock to react in the presence of the molecular sieve catalyst, to form a product stream comprising ethylene, propylene and C₄ hydrocarbon; iii) withdrawing the product stream from the top of the reactor, and passing it to a separation system, to separate ethylene, propylene and C₄ hydrocarbon; and

iv) circulating the C₄ hydrocarbon separated in step iii) back to step i).

In an embodiment of the present invention, the C₄ hydrocarbon comprised in the feedstock of step i) includes mixed C₄ hydrocarbon from other petroleum chemical processes such as steam cracking or catalytic cracking in addition to the C₄ hydrocarbon separated in step iii). In an embodiment of the present invention, a portion of feedstock is fed to the reactor from the distributor at the bottom of the reactor, and another portion of feedstock is fed to the reactor from one location above the distributor. In another embodiment of the present invention, a portion of feedstock is fed to the reactor from the distributor at the bottom of the reactor, and another portion of feedstock is fed to the reactor from multiple locations spaced horizontally and/or vertically above the distributor. In these two embodiments, the feedstock streams fed to the reactor from the bottom distributor and the individual injection ports may have the same or different composition. For example, it is possible that the C₄ hydrocarbon is mixed with at least one of methanol and dimethyl ether, and then the mixture is fed to the reactor from the distributor at the bottom of the reactor and from the one or more locations above the distributor. Alternatively, it is possible that methanol and/or dimethyl ether is fed to the reactor from the distributor at the bottom of the reactor, and the C₄ hydrocarbon is fed to the reactor from the one or more locations above the distributor. Alternatively, it is possible that a portion of methanol and/or dimethyl ether is fed to the reactor from the distributor at the bottom of the reactor, and the C₄ hydrocarbon and the remaining methanol and/or dimethyl ether are fed to the reactor from the one or more locations above the distributor. Alternatively, it is possible that the C₄ hydrocarbon is fed to the reactor from the distributor at the bottom of the reactor, and methanol and/or dimethyl ether are/is fed to the reactor from the one or more locations above the distributor. Alternatively, it is possible that a portion of C₄ hydrocarbon is fed to the reactor from the distributor at the bottom of the reactor, and the remaining C₄ hydrocarbon as well as methanol and/or dimethyl ether is fed to the reactor from the one or more locations above the distributor. In these two embodiments, a weight ratio of the feedstock fed to the reactor from the distributor at the bottom of the reactor to the feedstock fed to the reactor from the one or more locations above the distributor may be in a range of from 1:3 to 20:1, preferably from 1:2 to 15:1, more preferably from 1:1.5 to 10:1, and most preferably from 1:1 to 8:1.

If one or more injection ports above the distributor are employed, their locations may vary in a broad range along the axis direction of the reactor, but in generally in a range of from 1/10 to ⅘, preferably from ⅕ to ⅗, and more preferably from ⅕ to ½ reaction zone height above the distributor at the reactor bottom. If multiple injection ports spaced along the axis direction of the reactor are employed, the number of the injection ports may vary broadly. However, overmuch injection ports not only increase complicacy of the equipment but also inconvenience the maintenance, even affect the flow behavior of reagents in the reaction zone. In addition, when the number of the injection ports spaced along the axis direction of the reactor increases to a certain level or the location of an injection port is too high, the conversion of the feedstock may decrease to an unacceptable level. Thus, the number of the injection ports spaced along the axis direction of the reactor is generally not more than 4. If multiple injection ports spaced horizontally on the wall of the reactor are employed, the number of the injection ports may vary broadly but is generally not more than 4. The number and location of the injection port should be suitably set under the precondition that the conversion of the feedstock is acceptable. The amount of feedstock fed from individual injection ports may be the same or different.

Optionally, any portion of the feed in the method of the invention may comprise a diluent known by those skilled in the art, such as C₁ to C₃ alkanes, for example methane, ethane, propane; C₂ to C₄ alcohols, for example ethanol n-propanol, iso-propanol, n-butanol and iso-butanol; ethers, for example those having 3 to 8 carbon atoms; CO; CO₂; nitrogen; steam; and monocyclic arenes, for example benzene and toluene. As used in the description and the appended claims, the term diluent does not include C₄ hydrocarbon.

In principle, the method of the present invention may employ any catalytic reactor known in the art, such as dense phase fluidized-bed reactors, fast fluidized-bed reactors, riser reactors, moving-bed reactors and fixed-bed reactors. However, considering that the molecular sieve catalysts used in the method of the present invention have a characteristic that they are quickly deactivated, it is preferred to employ various dynamic bed reactors, such as fluidized-bed reactors, moving-bed reactors, riser reactors, and the like. Fast fluidized-bed reactors are particularly preferred. By using such dynamic bed reactors, continuous catalyst regeneration and circulation can be achieved. The method of the present invention may be performed in a single reactor or in multiple reactors parallel or in series.

In an embodiment of the present invention, the method of the present invention may employ the following process conditions: a reaction temperature inside the reaction zone ranging from 350 to 600° C., preferably from 400 to 600° C., more preferably from 400 to 550° C., and most preferably from 450 to 550° C.; a total weight hourly space velocity (WHSV) of methanol and/or dimethyl ether ranging from 0.5 to 100 h⁻¹, preferably from 1 to 50 h⁻¹, and more preferably from 1.5 to 20 h⁻¹; a gas superficial linear velocity inside the reaction zone ranging from 0.1 to 10 m/s, preferably from 0.8 to 5 m/s, and more preferably from 1 to 2 m/s; and a volume ratio of C₄ hydrocarbon to methanol or dimethyl ether or the sum of the both (if both methanol and dimethyl ether are used) in the feedstock of step i) ranging from 0.1:1 to 1:1, and preferably from 0.1:1 to 0.5:1.

The molecular sieve catalyst useful in the method of the invention may be any of molecular sieve catalysts known by those skilled in the art to be suitable for MTO process. In a preferred embodiment, the molecular sieve catalyst comprises one or more selected from the group consisting of ZSM molecular sieves and SAPO molecular sieves, more preferably ZSM-5 and/or SAPO-34 molecular sieve, and most preferably SAPO-34 molecular sieve. The catalyst comprises optionally a matrix known by those skilled in the art, such as silica, alumina, titania, zirconia, magnesia, thoria, silica-alumina, various clays, and mixtures thereof. The techniques to prepare a suitable molecular sieve catalyst are known by those skilled in the art.

The separation of the product stream may be accomplished by any technique known per se.

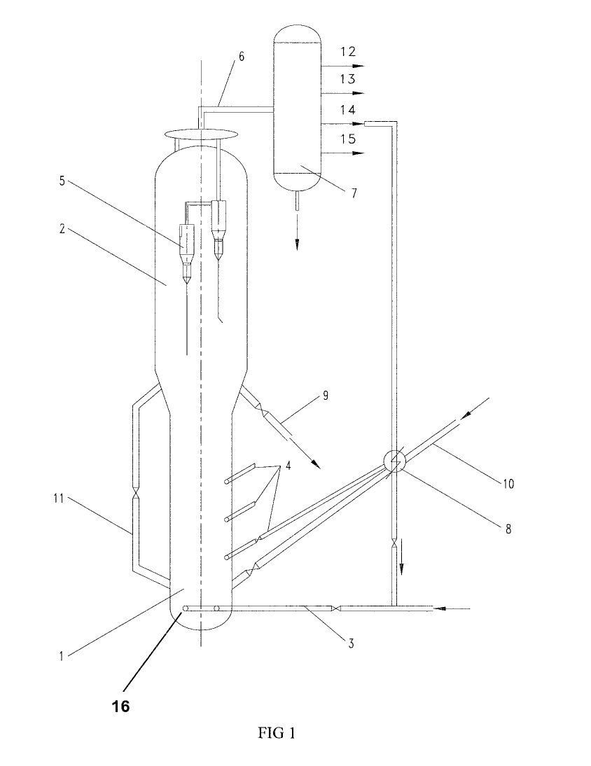

With reference to FIG. 1, an embodiment of the present invention will be described below, wherein a fast fluidized-bed reactor is used, and reagents are fed to the reactor from a distributor at the bottom of the reactor and three injection ports above the distributor. However, the method of the present invention may also employ a reactor of other types mentioned above, for example, a dense phase fluidized-bed reactor, and other feeding mode mentioned above, for example, a mode that reaction feedstock is fed to the reactor from the distributor at the bottom of the reactor and from one injection port above the distributor. As shown in FIG. 1, a first portion of feed is fed from the bottom of the reactor via line 3 and distributor 16 into reaction zone 1 containing a molecular sieve catalyst. The distributor 16 may be in the form of nozzle, porous distribution plate, tube distributor, or the like. The first portion of feed is fed at least partially in gas state into the reaction zone 1, to maintain the catalyst in the reaction zone 1 in fluidizing state. A second portion of feed is fed into the reaction zone 1 via three injection ports 4 spaced along the axis direction of the reactor. The first portion of feed and/or the second portion of feed may be heat exchanged with a catalyst carrying an amount of heat, and enter(s) the reaction zone 1 after having been heated to a desired temperature. The catalyst carrying an amount of heat may be one in the transporting line from the reactor to a regenerator (not shown) or from the regenerator to the reactor.

The first portion of feed and the second portion of feed contact with the catalyst and react in the reaction zone 1, to form a product stream containing ethylene, propylene and C₄ hydrocarbon. The product stream entraining some of catalyst enters upwards a gas-solid separation zone 2, where it is separated by a cyclone 5 located therein into a gaseous product stream and a solid catalyst stream. The gaseous product stream enters subsequent separation stage 7 via outlet line 6, to be separated into ethylene stream 12, propylene stream 13, C₄ hydrocarbon stream 14 and other component stream 15 by a process well known by those skilled in the art. The C₄ hydrocarbon stream 14 is subjected to heat exchange in a heat exchanger 8 with the catalyst from a regenerator, and then fed into the reactor 1 via the distributor 16 and/or the injection ports 4. The solid catalyst separated by the cyclone 5 is collected in the lower portion of the separation zone 2. The solid catalyst in the lower portion of the separation zone 2 may be circulated to the reaction zone 1 via a catalyst return 11 or sent to the regenerator via a line 9 to be regenerated. The regenerated catalyst is returned to the reaction zone 1 via a line 10. The amount of the catalyst returned into the reaction zone 1 via the catalyst return 11 and the amount of the catalyst returned to the reaction zone 1 from the regenerator via the line 10, and/or the regeneration extent of the catalyst can be adjusted to suitably adjust the average amount of coke on the catalyst in the reaction zone 1, thereby to adjust the selectivity of reaction in the reaction zone. Catalyst regeneration processes are known by those skilled in the art, for example one by burning off coke in an oxygen-containing atmosphere. Prior to the regeneration, the coked catalyst withdrawn from the reactor is optionally stripped, to recover volatile carbonaceous material adsorbed thereon.

In the method of the present invention, the reaction of converting methanol and/or dimethyl ether to light olefins and the reaction of catalytically cracking mixed C₄ hydrocarbon to form ethylene and propylene are simultaneously carried out. The mixed C₄ hydrocarbon functions as a diluent, favoring the enhancement of the selectivity to ethylene and propylene in the conversion reaction of methanol and/or dimethyl ether. Furthermore, the method of the present invention utilizes the C₄ hydrocarbon formed in the conversion of methanol and/or dimethyl ether to produce ethylene and propylene, and thus enhances the yield of ethylene and propylene in MTO process as a whole.

By using the method of the present invention, it is possible to achieve a total yield of ethylene and propylene of up to 39% by weight.

### EXAMPLES

The following examples are given for further illustrating the invention, but do not make limitation to the invention in any way.

In the following examples, methanol conversion and dimethyl ether conversion means:

% methanol conversion=((inlet methanol mass flow rate−outlet methanol mass flow rate)/inlet methanol mass flow rate)×100, and

% dimethyl ether conversion=((inlet dimethyl ether mass flow rate−outlet dimethyl ether mass flow rate)/inlet dimethyl ether mass flow rate)×100.

In the following examples, ethylene yield and propylene yield means:

% ethylene yield=(outlet ethylene mass flow rate/inlet total mass flow rate of methanol and dimethyl ether)×100, and

% propylene yield=(outlet propylene mass flow rate/inlet total mass flow rate of methanol and dimethyl ether)×100.

### Examples 1 to 42

In a mini fast fluidized-bed reactor, experiments were carried out by using a SAPO-34 molecular sieve catalyst molded by spray drying comprising 50 wt % of SAPO-34 molecular sieve and 50 wt % of alumina matrix. Temperature inside the reaction zone was 500° C., a WHSV of methanol and/or dimethyl ether (DME) was 1.5 h⁻¹, gas superficial linear velocity in the reaction zone was 2 m/s, and reaction pressure was 0.01 MPa (gauge). Mixed C₄ hydrocarbon had a composition shown in Table 1. Methanol, DME and the C₄ hydrocarbon were fed into the reactor in different proportions and feeding modes (shown in Table 2), to contact with the catalyst and react. Reaction product was analyzed by an in-line gas chromatogragh. The results obtained when the experiments had been run for 10 min are shown in Table 2.

TABLE 1
Composition of mixed C4 hydrocarbon

| Component | Content, wt % |
|---|---|
| Isobutane | 0.31 |
| n-Butane | 5.07 |
| Trans 2-butene | 35.82 |
| 1-Butene | 25.62 |
| Isobutene | 5.37 |
| Cis 2-butene | 26.44 |
| 1,3-Butadiene | 1.37 |

TABLE 2

| | | | | Volume ratio of C₄ | Total | | |
|---|---|---|---|---|---|---|---|
| | | | | hydrocarbon | conversion | | |
| | | | | to methanol | of methanol | | |
| | | | Distance between | or dimethyl ether | and/or | | |
| | | | injection port and | or the both (if both | DME (if DME | Ethylene | Propylene |
| Example | Feed via | Feed via injection port | distributor/reaction | methanol and dimethyl | was used) | yield | yield |
| No. | distributor | above distributor | zone height | ether were used) | wt % | wt % | wt % |
| 1 | methanol | C4 | 1/2 | 0.1:1 | 100 | 21.3 | 14.4 |
| 2 | methanol | methanol + C4 | | | 99.8 | 21.9 | 14.8 |
| 3 | methanol | DME + C4 | | | 99.8 | 21.7 | 14.2 |
| 4 | methanol | methanol + DME + C4 | | | 99.7 | 21.8 | 14.5 |
| 5 | methanol + DME | methanol + C4 | 1/2 | 0.2:1 | 99.9 | 20.2 | 15.8 |
| 6 | methanol + DME | DME + C4 | | | 99.9 | 20.3 | 15.7 |
| 7 | methanol + DME | methanol + DME + C4 | | | 99.8 | 20.5 | 16 |
| 8 | methanol + DME | C4 | | | 99.8 | 20.1 | 15.8 |
| 9 | DME | C4 | 1/3 | 0.3:1 | 99.7 | 21.4 | 15.9 |
| 10 | DME | methanol + C4 | | | 99.6 | 21.6 | 16.1 |
| 11 | DME | methanol + DME + C4 | | | 99.6 | 21.5 | 16.4 |
| 12 | DME | DME + C4 | | | 99.7 | 21.8 | 16.2 |
| 13 | methanol + C4 | methanol + C4 | 1/3 | 0.3:1 | 99.6 | 21.5 | 15.7 |
| 14 | methanol + C4 | C4 | | | 99.9 | 22.1 | 15.6 |
| 15 | methanol + C4 | methanol | | | 99.4 | 22.4 | 15.5 |
| 16 | methanol + C4 | DME + C4 | | | 99.4 | 22.3 | 15.2 |
| 17 | methanol + C4 | DME | | | 99.5 | 21.9 | 15.5 |
| 18 | methanol + C4 | methanol + DME + C4 | | | 99.5 | 21.3 | 15.8 |
| 19 | methanol + C4 | methanol + DME | | | 99.2 | 21.2 | 15.1 |
| 20 | methanol + C4 | | | | 100 | 20.2 | 14.8 |
| 21 | C4 | methanol | 1/2 | 0.4:1 | 99.8 | 21.3 | 16.8 |
| 22 | C4 | DME | | | 99.6 | 20.9 | 16.7 |
| 23 | C4 | methanol + C4 | | | 99.9 | 21.7 | 17.1 |
| 24 | C4 | DME + C4 | | | 99.5 | 21.5 | 17 |
| 25 | C4 | methanol + DME + C4 | | | 99.4 | 21.8 | 17.2 |
| 26 | C4 | methanol + DME | | | 99.6 | 21.6 | 16.9 |
| 27 | DME + C4 | methanol | 1/2 | 0.5:1 | 99.7 | 20.8 | 17.3 |
| 28 | DME + C4 | DME | | | 99.6 | 20.6 | 17.2 |
| 29 | DME + C4 | methanol + C4 | | | 99.4 | 20.7 | 17.2 |
| 30 | DME + C4 | DME + C4 | | | 99.4 | 20.6 | 17 |
| 31 | DME + C4 | methanol + DME | | | 99.3 | 20 | 17.2 |
| 32 | DME + C4 | methanol + DME + C4 | | | 99.4 | 20.4 | 16.9 |
| 33 | DME + C4 | C4 | | | 99.8 | 20.1 | 17.4 |
| 34 | DME + C4 | | | | 99.8 | 19.9 | 17.3 |
| 35 | methanol + DME + C4 | methanol | 1/4 | 1.0:1 | 99.6 | 18.9 | 18.5 |
| 36 | methanol + DME + C4 | DME | | | 99.5 | 18.4 | 18.7 |
| 37 | methanol + DME + C4 | C4 | | | 99.8 | 18.1 | 19.1 |
| 38 | methanol + DME + C4 | methanol + DME + C4 | | | 99.5 | 18.3 | 19.2 |
| 39 | methanol + DME + C4 | methanol + DME | | | 99.4 | 18.8 | 18.9 |
| 40 | methanol + DME + C4 | methanol + C4 | | | 99.5 | 18.7 | 19.2 |
| 41 | methanol + DME + C4 | DME + C4 | | | 99.5 | 18.7 | 19.3 |
| 42 | methanol + DME + C4 | | | | 99.9 | 17.9 | 18.9 |

Note:
1. If both methanol and DME were used as feedstock, then the volume ratio of methanol to DME was 1:1.
2. If methanol feedstock was fed from the distributor and the injection port above the distributor, then 50% of methanol was fed from the distributor and 50% of methanol was fed from the injection port above the distributor.
3. If DME feedstock was fed from the distributor and the injection port above the distributor, then 50% of DME was fed from the distributor and 50% of DME was fed from the injection port above the distributor.
4. If C4 hydrocarbon was fed from the distributor and the injection port above the distributor, then 50% of C4 hydrocarbon was fed from the distributor and 50% of C4 hydrocarbon was fed from the injection port above the distributor.

### Example 43

In a mini moving-bed reactor, an experiment was carried out by using a 20 to 40 mesh ZSM-34 molecular sieve catalyst comprising 50 wt % of the molecular sieve and 50 wt % of alumina matrix. Reaction temperature was 550° C., a volume ratio of mixed C₄ hydrocarbon (having a composition shown in Table 1) to dimethyl ether was 0.1:1, a WHSV of dimethyl ether was 20 h⁻¹, gas superficial linear velocity in the reaction zone was 5 m/s, and reaction pressure was 0.01 MPa (gauge). The feed consisting of dimethyl ether and C₄ hydrocarbon was fed into the reactor from a porous distribution plate at the bottom of the reactor, to contact with the catalyst and react. Reaction product was analyzed by an in-line gas chromatogragh. The results obtained when the experiment had been run for 10 min are as follows: dimethyl ether conversion is 97.5 wt %, ethylene yield is 17.3 wt %, and propylene yield is 7.1 wt %.

### Example 44

An experiment was conducted according to the procedure as described in Example 20, except that the reactor was a dense phase fluidized-bed reactor, reaction temperature was 350° C., the WHSV of methanol was 0.5 h⁻¹, and gas superficial linear velocity in the reaction zone was 0.1 m/s. The results obtained when the experiment had been run for 10 min are as follows: methanol conversion is 98.4 wt %, ethylene yield is 13.1 wt %, and propylene yield is 13.3 wt %.

### Example 45

An experiment was conducted according to the procedure as described in Example 20, except that the reactor was a riser reactor, reaction temperature was 600° C., the WHSV of methanol was 100 h⁻¹, gas superficial linear velocity in the reaction zone was 10 m/s, and the volume ratio of the mixed C₄ hydrocarbon to the methanol was changed to 0.7:1. The results obtained when the experiment had been run for 10 min are as follows: methanol conversion is 100 wt %, ethylene yield is 18.7 wt %, and propylene yield is 12.8 wt %.

### Example 46

An experiment was conducted according to the procedure as described in Example 43, except that the WHSV of dimethyl ether was 50 h⁻¹, gas superficial linear velocity in the reaction zone was 1 m/s, and a 20 to 40 mesh ZSM-5 molecular sieve catalyst comprising 50 wt % of the molecular sieve and 50 wt % of alumina matrix was used as catalyst. The results obtained when the experiment had been run for 10 min are as follows: methanol conversion is 100 wt %, ethylene yield is 8.6 wt %, and propylene yield is 16.9 wt %.

### Example 47

An experiment was conducted according to the procedure as described in Example 1, except that the WHSV of methanol was 1 h⁻¹, gas superficial linear velocity in the reaction zone was 0.8 m/s, methanol was fed to the reaction zone from the bottom distributor, and the mixed C₄ hydrocarbon was fed to the reaction zone from one injection port on the wall of the reactor, which was ⅓ reaction zone height away from the bottom distributor. The results obtained when the experiment had been run for 10 min are as follows: methanol conversion is 100 wt %, ethylene yield is 20.9 wt %, and propylene yield is 16.7 wt %.

### Example 48

An experiment was conducted according to the procedure as described in Example 47, except that the volume ratio of the mixed C₄ hydrocarbon to the methanol was 0.8:1. The results obtained when the experiment had been run for 10 min are as follows: methanol conversion is 99.7 wt %, ethylene yield is 19.2 wt %, and propylene yield is 19.3 wt %.

### Example 49

An experiment was conducted according to the procedure as described in Example 47, except that a SAPO-18 molecular sieve catalyst molded by spray drying comprising 50 wt % of the molecular sieve and 50 wt % of alumina matrix was used as catalyst, and the volume ratio of the mixed C₄ hydrocarbon to the methanol was 1:1. The results obtained when the experiment had been run for 10 min are as follows: methanol conversion is 97.4 wt %, ethylene yield is 19.1 wt %, and propylene yield is 15.0 wt %.

### Example 50

An experiment was conducted according to the procedure as described in Example 20, except that the volume ratio of mixed C₄ hydrocarbon to methanol in the feedstock was 1:1, the mixed C₄ hydrocarbon was fed to the reaction zone via the distributor at reactor bottom, and the methanol was fed to the reaction zone from four injection ports on the wall of the reactor, which were ⅛ reaction zone height, ⅙ reaction zone height, ¼ reaction zone height and ½ reaction zone height away from the bottom distributor, respectively. The results obtained when the experiment had been run for 10 min are as follows: methanol conversion is 93.5 wt %, ethylene yield is 20.6 wt %, and propylene yield is 17.9 wt %.

### Example 51

An experiment was conducted according to the procedure as described in Example 20, except that the volume ratio of mixed C₄ hydrocarbon to methanol in the feedstock was 1:1, and 50 wt % of the mixed C₄ hydrocarbon and methanol were fed to the reaction zone via the distributor at reactor bottom, and the remaining mixed C₄ hydrocarbon was fed to the reaction zone from two injection ports on the wall of the reactor, which were ¼ reaction zone height and ½ reaction zone height away from the bottom distributor, respectively. The results obtained when the experiment had been run for 10 min are as follows: methanol conversion is 98.8 wt %, ethylene yield is 17.9 wt %, and propylene yield is 18.7 wt %.

### Example 52

An experiment was conducted according to the procedure as described in Example 20, except that the reaction temperature was changed to 450° C. The results obtained when the experiment had been run for 10 min are as follows: methanol conversion is 99.2 wt %, ethylene yield is 18.4 wt %, and propylene yield is 16.7 wt %.

### Example 53

An experiment was conducted according to the procedure as described in Example 20, except that the reaction temperature was changed to 400° C. The results obtained when the experiment had been run for 10 min are as follows: methanol conversion is 97.1 wt %, ethylene yield is 16.2 wt %, and propylene yield is 17.1 wt %.

### Comparative Example 1

An experiment was conducted according to the procedure as described in Example 20, except that the feed was changed to pure methanol feed. The results obtained when the experiment had been run for 10 min are as follows: methanol conversion is 100 wt %, ethylene yield is 19.2 wt %, and propylene yield is 13.2 wt %.

### Comparative Example 2

An experiment was conducted according to the procedure as described in Example 20, except that the feed was changed to methanol and steam feed, and the weight ratio of steam to methanol was 0.25:1. The results obtained when the experiment had been run for 10 min are as follows: methanol conversion is 100 wt %, ethylene yield is 20.3 wt %, and propylene yield is 12.8 wt %.

While the invention has been described with reference to exemplary embodiments, it will be understood by those skilled in the art that various changes and modifications may be made without departing from the spirit and scope of the invention. Therefore, the invention is not limited to the particular embodiments disclosed as the best mode contemplated for carrying out this invention, but the invention will include all embodiments falling within the scope of the appended claims.

1. A method for enhancing yields of ethylene and propylene in MTO process, comprising:
i) feeding a feedstock comprising C₄ hydrocarbon and at least one of methanol and dimethyl ether from a distributor at the bottom of a reactor and optionally from at least one location above the distributor into a reaction zone containing a molecular sieve catalyst;
ii) allowing the feedstock to react in the presence of the molecular sieve catalyst, to form a product stream comprising ethylene, propylene and C₄ hydrocarbon;
iii) withdrawing the product stream from the top of the reactor, and passing it to a separation system, to separate ethylene, propylene and C₄ hydrocarbon; and
iv) circulating the C₄ hydrocarbon separated in step iii) back to step i).

2. The method of claim 1, wherein the C₄ hydrocarbon comprised in the feedstock of step i) includes mixed C₄ hydrocarbon from other petroleum chemical processes such as steam cracking or catalytic cracking in addition to the C₄ hydrocarbon separated in step iii).

3. The method of claim 1, wherein the feedstock comprising C₄ hydrocarbon and at least one of methanol and dimethyl ether is fed to the reaction zone containing the molecular sieve catalyst from the distributor at the bottom of the reactor and from at least one injection port above the distributor.

4. The method of claim 3, wherein the feedstock fed to the reactor from the bottom distributor and the injection port(s) have the same or different composition.

5. The method of claim 3, wherein the C₄ hydrocarbon is mixed with at least one of methanol and dimethyl ether, and then the mixture is fed to the reactor from the distributor at the bottom of the reactor and one or more locations above the distributor.

6. The method of claim 3, wherein methanol and/or dimethyl ether is fed to the reactor from the distributor at the bottom of the reactor, and the C₄ hydrocarbon is fed to the reactor from the one or more locations above the distributor.

7. The method of claim 3, wherein a portion of methanol and/or dimethyl ether is fed to the reactor from the distributor at the bottom of the reactor, and C₄ hydrocarbon and the remaining methanol and/or dimethyl ether are fed to the reactor from the one or more locations above the distributor.

8. The method of claim 3, wherein the C₄ hydrocarbon is fed to the reactor from the distributor at the bottom of the reactor, and methanol and/or dimethyl ether are/is fed to the reactor from the one or more locations above the distributor.

9. The method of claim 3, wherein a portion of C₄ hydrocarbon is fed to the reactor from the distributor at the bottom of the reactor, and the remaining C₄ hydrocarbon as well as methanol and/or dimethyl ether is fed to the reactor from the one or more locations above the distributor.

10. The method of claim 3, wherein a weight ratio of the feedstock fed to the reactor from the distributor at the bottom of the reactor to the feedstock fed to the reactor from the one or more locations above the distributor is in a range of from 1:3 to 20:1.

11. The method of claim 1, wherein the reactor is a dense phase fluidized-bed reactor, a fast fluidized-bed reactor, a riser reactor, a moving-bed reactor or a fixed-bed reactor.

12. The method of claim 11, wherein the reactor is a fast fluidized-bed reactor.

13. The method of claim 1, wherein the method is carried out under the following conditions: a reaction temperature inside the reaction zone ranging from 350 to 600° C., a total WHSV of methanol and/or dimethyl ether ranging from 0.5 to 100 h⁻¹, a gas superficial linear velocity in the reaction zone ranging from 0.1 to 10 m/s, and a volume ratio of C₄ hydrocarbon to methanol or dimethyl ether or the sum of the both, if both methanol and dimethyl ether is used, in step i) ranging from 0.1:1 to 1:1.

14. The method of claim 13, wherein the method is carried out under the following conditions: a reaction temperature inside the reaction zone ranging from 450 to 550° C., a total WHSV of methanol and/or dimethyl ether ranging from 1 to 50 h⁻¹, a gas superficial linear velocity in the reaction zone ranging from 0.8 to 5 m/s, and a volume ratio of C₄ hydrocarbon to methanol or dimethyl ether or the sum of the both, if both methanol and dimethyl ether is used, in step i) ranging from 0.1:1 to 0.5:1.

15. The method of claim 1, wherein the catalyst comprises one or more selected from the group consisting of ZSM molecular sieves and SAPO molecular sieves.

16. The method of claim 15, wherein the catalyst comprises ZSM-5 molecular sieve and/or SAPO-34 molecular sieve.

17. The method of claim 15, wherein the catalyst further comprises a matrix.

18. The method of claim 3, wherein the reactor has no more than 4 injection ports spaced vertically or horizontally on its wall.

19. The method of claim 1, wherein the feedstock of step i) further comprises a diluent.

20. The method of claim 19, wherein the diluent is selected from the group consisting of C₁ to C₃ alkanes, C₂ to C₄ alcohols, ethers having 3 to 8 carbon atoms, CO, CO₂, nitrogen, steam, benzene and toluene.
